(19) United States
(12) Patent Application Publication
(10) Pub. No.: US 2009/0163923 A1
(43) Pub. Date: Jun. 25, 2009

(54) SURGICAL JIG AND METHODS OF USE

(76) Inventors: Magnus Flett, Wakefield (GB); Gary Fenton, Huddersfield (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/598,287

(22) PCT Filed: Feb. 11, 2005
(86) PCT No.: PCT/GB05/00484

§ 371 Date: Jun. 13, 2007

(30) Foreign Application Priority Data
Feb. 27, 2004 (GB) 0404345.1

(51) Int. Cl. A61B 17/58

(52) U.S. Cl. 606/89; 606/87

(57) ABSTRACT

A surgical jig defines an axis relative to a body part and includes a support, a first guide element mounted on the support and a second guide element mounted on the support. Each guide element includes a guide channel and the guide channels between them define a substantially linear jig axis.

The present invention relates to a surgical apparatus and methods of use, and in particular to a surgical jig for defining an axis, methods of use of the jig, computer aided surgical systems and methods, computer code and computer program products.

During surgical procedures a surgeon can need to determine an axis relative to a body part, so as to accurately locate an instrument or tool, to locate the correct position and/or direction at which to make an incision, drill a hole, insert an instrument, tool or implant or merely to determine the position and orientation of an axis in relation to a body part. For example in cranial surgery, in can be necessary to drill into the skull and it can be important to accurately determine the initial entry point and direction of drilling in order to ensure that important brain structures are not harmed during the procedure. In orthopaedic surgical procedures, for example a hip replacement operation, it can be necessary to accurately determine the axis of the femoral neck in order to correctly position a femoral head implant.

Irrespective of the particular surgical application, a guide which enables a surgeon to accurately and reliably determine an axis relative to a body part would be beneficial.

According to a first aspect of the present invention, there is provided a surgical jig for defining an axis relative to a body part. The jig comprises a support, a first guide element and a second guide element. The first guide element can have a first guide channel, can be mounted on the support and can be translatable over a first plane. The second guide element can have a second guide channel, can be mounted on the support and can be translatable over a second plane. The second plane can be substantially parallel to the first plane. The first guide channel and second guide channel define a substantially linear jig axis.

Hence, the movable guides can be used to define a jig axis by translating them over parallel planes.

The guide channels can be closed bores or can have an at least partially open structure. e.g. C or U shaped. The guide channels can be configure to receive and instrument or tool in use. The guide channels can provide a cannulated guide for a tool or instrument. The guide channels can act as a bushing for a drill bit or part of a drill. The jig can include a drive mechanism. The drive mechanism can be operable to move the first guide element and/or to move the second guide element. A separate drive mechanism can be provided for each guide element.

The support can have a generally frame like construction. This provides an open structure so that the position of the guide elements can remain visible during use.

The or each drive mechanism can include a carrier for bearing one of the guide elements. The or each drive mechanism can include first and second carriers bearing the first guide element. The carriers can be parallel to the first plane and perpendicular to each other.

The drive mechanism can include a motor actuable to drive the carrier to control the position of the first guide element over the plane. The motor can be an electric motor. In other embodiments, the motor can be a hydraulic motor, a pneumatic motor or a linear motor.

The support can include a first pair of opposed sides and a second pair of opposed sides. Each side can include a slider or slider mechanism. The second pair of sides can be oriented substantially perpendicularly to the first pair. A first carriage can extend between sliders of the first pair of sides and a second carriage can extend between sliders of the second pair of sides. The sliders can include a bushing. Each bushing can receive a part of a carriage. The part of the carriage can be journalled within the bushing.

The drive mechanism can include third and fourth carriers bearing the second guide element, disposed parallel to the second plane and perpendicular to each other. The drive mechanism can further include a motor actuable to drive the carriers to control the position of the second guide element over the plane.

The first pair of opposed sides can each include a further slider and the second pair of opposed sides can each include a further slider. The third carriage can extend between the further sliders of the first pair of sides and the fourth carriage can extend between the sliders of the second pair of sides.

The sliders can include a bushing. Each bushing can receive a part of a carriage. The part of the carriage can be journalled within the bushing. The bushing can be made of a low friction material, such as a plastics.

Each slider can includes a guide track. A bushing can be slidably mounted therein. An end of each carrier can be received in a respective bushing.

Each carrier can be a lead screw. Each carrier can be a chain, pulley or pneumatically or hydraulically actuable element. Each carrier can be independently driveable.

A separate motor can be provided for driving each carrier. Each motor can be an electric motor. Each motor can be a stepper motor. Each motor can be an indexed motor which can generate a signal indicating the degree of motion of what it drives.

The jig can be adapted to be tracked. The jig can further include a first marker detectable by a tracking system. The first marker can be attached to the jig so that the position of the jig in a reference frame can be determined.

The marker can be a wired or a wireless marker. The marker can reflect, or generate or transmit energy to a detector part of a tracking system. The energy can be electrical, electromagnetic or acoustic. The energy can be ultrasonic. The energy can be in the infrared, the radio frequency or microwave parts of the spectrum.

The jig can further include a second marker detectable by a tracking system. The second marker can be attached to the second guide element and the first marker can be attached to the first guide element. In this way, the position of the guide elements can be determined and hence the position of the jig axis can be derived. The jig can further include an instrument passing through the first guide channel and second guide channel. The first marker can be attached to the instrument. The instrument can have a substantially straight part, sufficiently long to pass through the first guide channel and second guide channel when inserted between them.

The jig can further comprising a first arm. The jig can further comprise a second arm. The first arm can connect the guide element to the support. The second arm can connect the second guide element to the support.

The first and second arms can be spaced along a longitudinal axis of the support. The first arm and/or the second arm can each be pivotally connected to the support. The first arm and/or the second arm can be pivotable about a longitudinal axis of the support. The first and/or second arm can be extendable along a longitudinal axis of the arm.

The jig can further comprising a base member. The base member can be pivotally attached to the support. The base member can include a formation for receiving a fastener to secure the guide to a bone. A part of the support can be journalled within the base member. The base member can clamp around the part of the support. Relative movement between the support and base member can be prevented when the base is secured to the bone by the fastener.

According to a further aspect of the invention, there is provided a computer aided surgical system for determining a linear axis relative to a body part. The system comprises a jig according to the first aspect of the invention and bearing detectable marker and a tracking system for determining the positions of the or each marker. The tracking system can produce marker position data. The system can also include a data processing device configured to operate on the marker position data and data representing the position of a predetermined axis to determine when the jig axis corresponds to the predetermined axis.

According to a further aspect of the invention, there is provided a method for defining an axis relative to a body part, using a surgical jig having a support, a first guide element having a first guide channel and a second guide element having a second guide channel. The method can comprise locating the surgical jig adjacent the body part and positioning the first guide at a first position and/or positioning the second guide at a second position. A jig axis is defined between the first and second guide channels. The first and second guide elements can be moved in respective substantially parallel planes.

The method can further comprise determining the position of a predetermined axis of the body part. The axis can correspond to an axis of a physical body part. The physical body part can be a bone or part of a bone, an organ or part of an organ, a brain structure or other body parts which have a particular geometry associated with them. The axis can merely be relative to a particular location of a body part, for example, an angle of incidence relative to a surgical site.

The method can further comprise moving the first and/or second guide elements until the jig axis is substantially co-linear with the predetermined axis. The first guide element and/or second guide element can be manually positioned. The first guide element and/or second guide element can be automatically positioned. The first guide element and/or second guide element can be driven by a motor.

The method can further comprise determining the position of the first guide element and the position of the second guide element. Hence the position of the jig axis can be determined from the guide element positions.

The position of the first guide element and the position of the second guide element can be determined by wirelessly tracking the first guide element and the second guide element.

The method can further comprise determining the position of the jig. The position of the jig can be determined by wirelessly tracking the jig. The position of the first guide element can be determined relative to the position of the jig and the position of the second guide element can be determined relative to the position of the jig.

The method can further comprise determining a current position of the jig axis. The current position of the jig axis can be determined from a current position of the first and/or second guide elements. Positional data can be generated representing the current position of the jig axis relative to the body. A visual representation of the position of the jig axis relative to the body can be generated. This provides an image guided surgery (IGS) based method for determining the jig axis position relative to the body.

The method can further comprise displaying an image of a body part together with the visual representation of the jig axis. The method can further comprise displaying a visual representation of a body axis together with the visual representation of the jig axis. The method can further comprise displaying a visual representation of the degree of alignment of the body axis with the jig axis.

The method can further comprise determining a current position of the jig axis based on current positions of the first and second guide elements. Positional data representing the current position of the jig axis can be generated and the position of a predetermined axis relative to the body can be determined. Control signals, instructions or data can be generated to control and/or drive the jig so as to reduce the separation between the position of the jig axis and the position of the pre-determined axis. This provides an automated method for aligning the jig axis and body axis.

The method can further comprise generating control signals, instructions or data to control and/or drive the jig until the jig axis and the position of the pre-determined axis are substantially co-linear.

A feed back loop can be used to correct the current jig axis position until it is co-linear with the body axis. The feedback loop can be provided by a tracking system. The feedback loop can be provided by a signal from a drive mechanism part of the jig.

According to a further aspect of the invention, there is provided computer program code executable by a data processing device to control a surgical jig having a support, a first guide element having a first guide channel and a second guide element having a second guide channel, the first guide channel and the second guide channel defining between them a jig axis. The computer program code can include instructions to generate data representing the position of a predetermined axis of a body part, determine the current positions of the first guide element and the second guide element, generate data representing the position of the Jig axis defined by a current position of the first guide element and second guide element, and generating control signals, instructions or data for driving or controlling the first and/or second guide elements to reduce the separation between the jig axis and the predetermined axis.

According to a yet further aspect of the invention, there is provided a computer readable medium bearing computer program code according to the preceding aspect of the invention.

The jig can include a plurality of feet engagable with a surface of the body part. The feet can have formations for receiving a fastening for securing the jig to a body part. The feet can be deformable so as to clamp about the body part. The feet can be hinged or otherwise pivotable. The feet can be of an elastic material which is resiliently deformable. The plurality of feet can be clamped about the body part to secure the jig to the body part. The feet can have formations adapted to engage with an anatomical feature of the body part so as to align or otherwise position the jig when mounted on the body part.

An embodiment of the invention will now be described, by way of example only, and with reference to the accompanying drawings, in which:

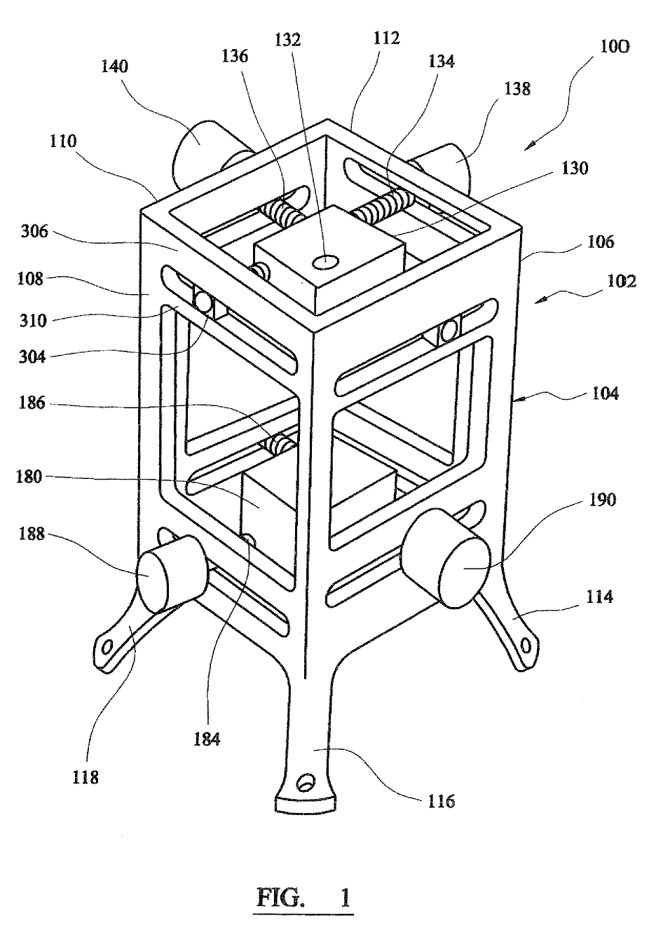

FIG. 1 shows a perspective view of a schematic illustration of a guide according to the invention;

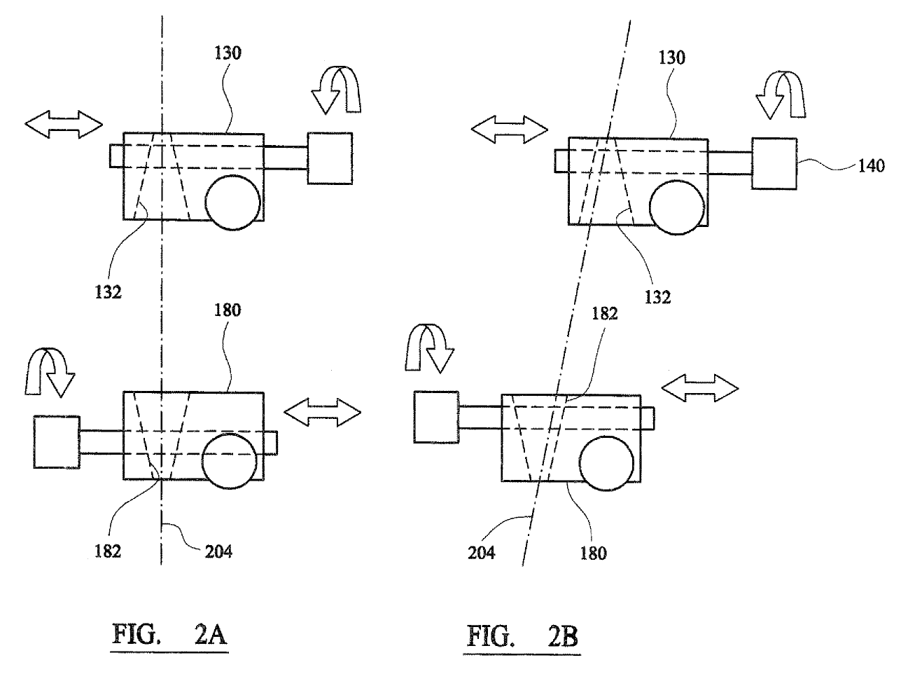

FIGS. 2A and 2B respectively show schematic illustrations of guide parts of the guide shown in FIG. 1 defining first and second axes;

FIG. 3 shows a schematic lateral cross section through a part of the guide shown in FIG. 1;

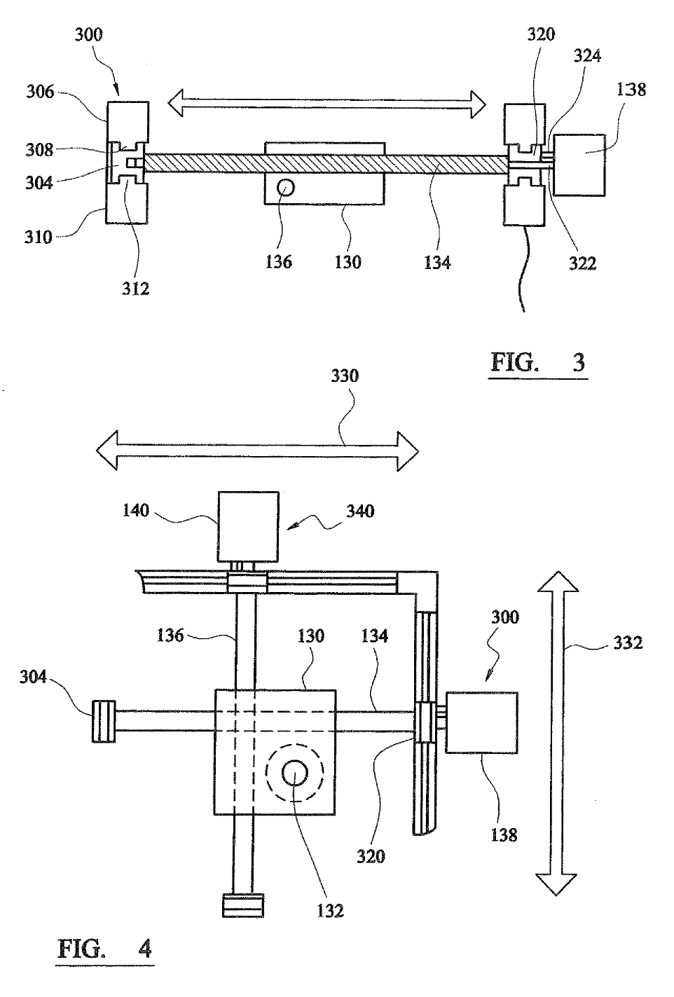

FIG. 4 shows a schematic transverse cross section through a part of the guide shown in FIG. 1;

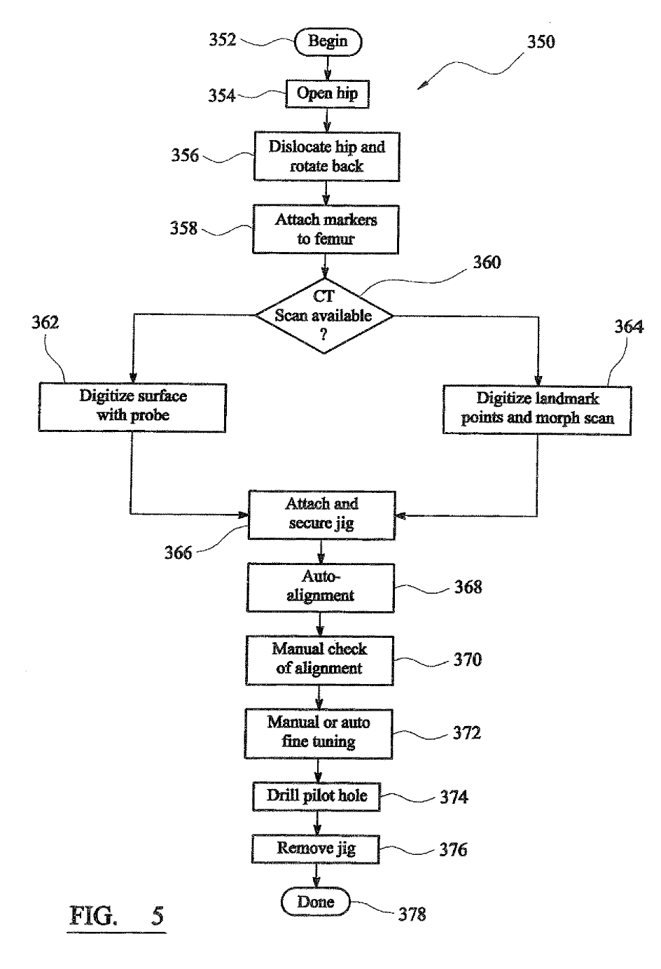

FIG. 5 shows a flow chart illustrating a surgical procedure using the guide according to the invention;

FIG. 6 shows a schematic block diagram of a computer aided surgical system including a guide according to the invention;

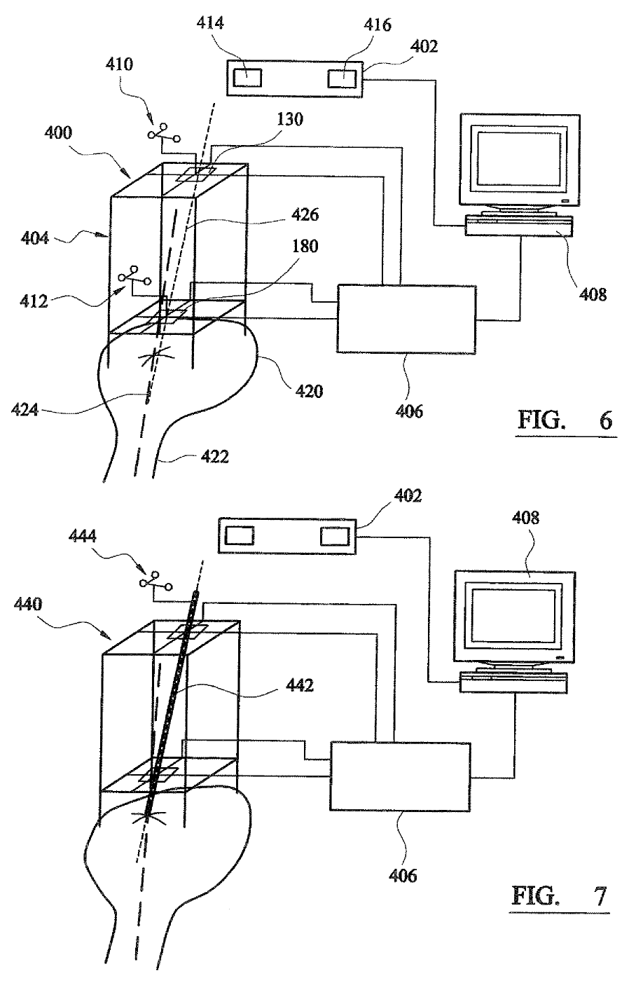

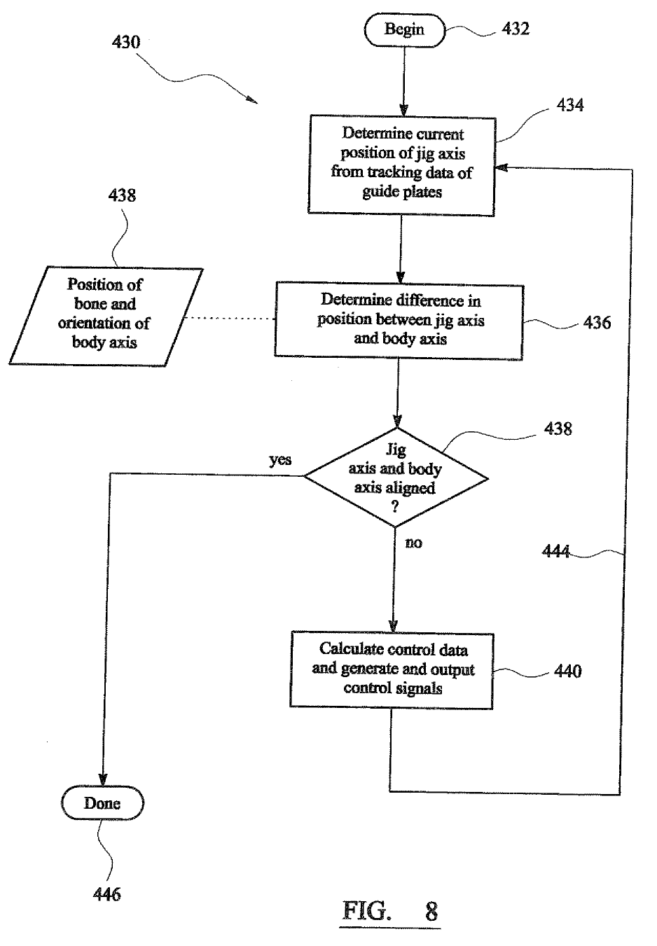

FIG. 7 shows a schematic block diagram of a further computer aided surgical system including a guide according to the invention;

FIG. 8 shows a flow chart illustrating a computer implemented method of automatically determining an axis according to the invention and using the systems shown in FIGS. 6 and 7;

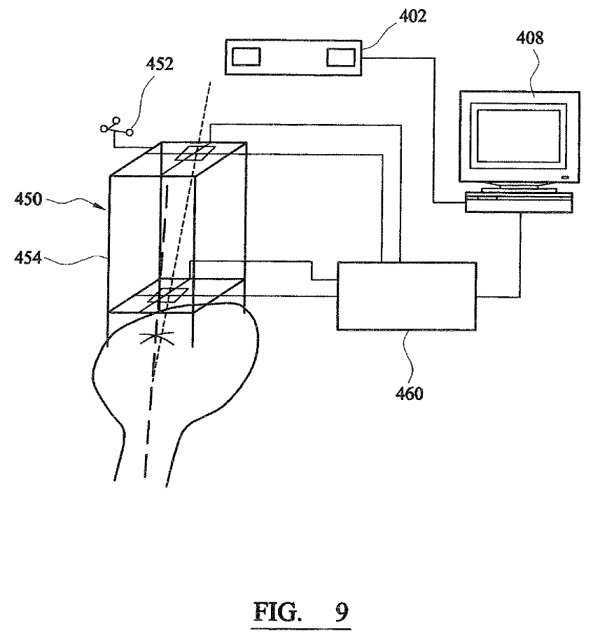

FIG. 9 shows a schematic block diagram of a further computer aided surgical system including a guide according to the invention;

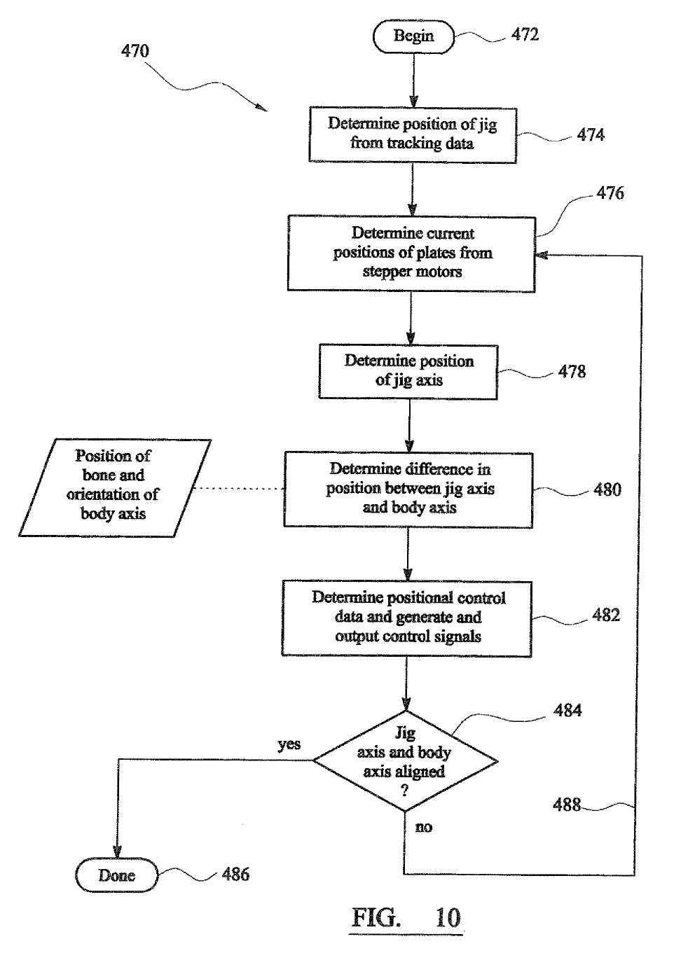

FIG. 10 shows a flow chart illustrating a further computer implemented method of automatically determining an axis according to the invention using the system shown in FIG. 9;

FIG. 11 shows a schematic perspective view of another guide according to the invention;

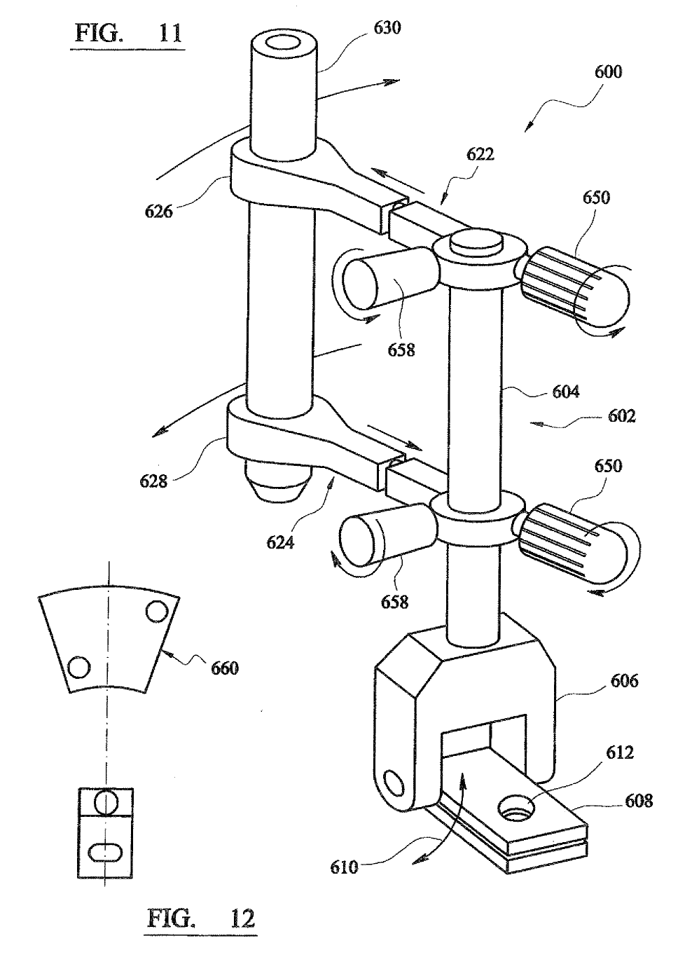

FIG. 12 shows a plan view illustrating the range of motion of the guide elements of the guide shown in FIG. 11;

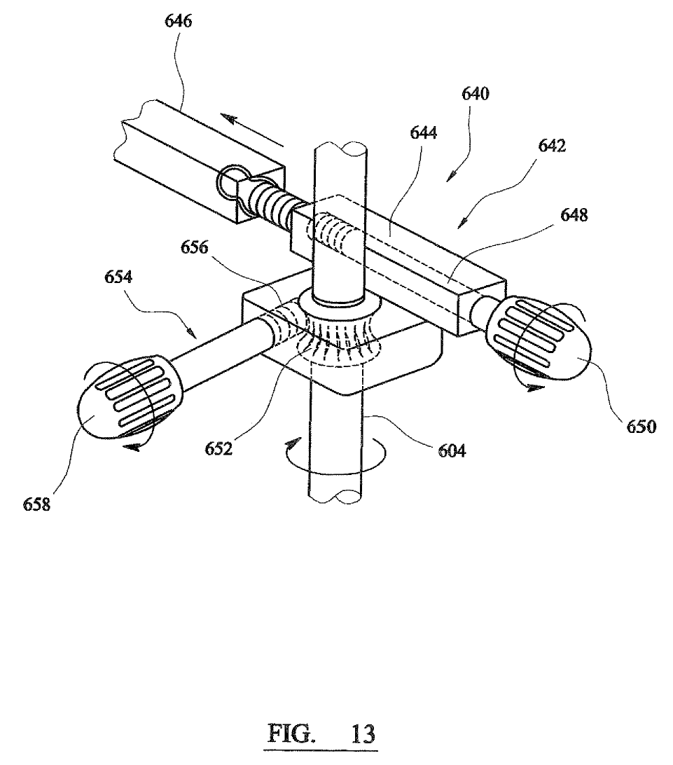

FIG. 13 shows a schematic perspective view of a part of the guide shown in FIG. 11;

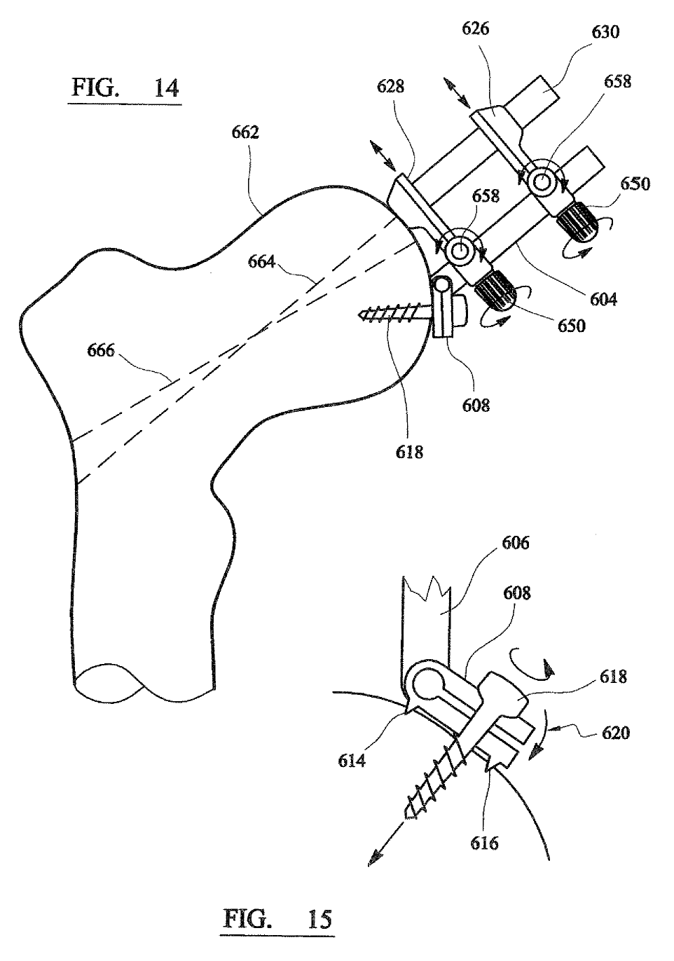

FIG. 14 shows a schematic side view of the guide in use mounted on the head of a femur;

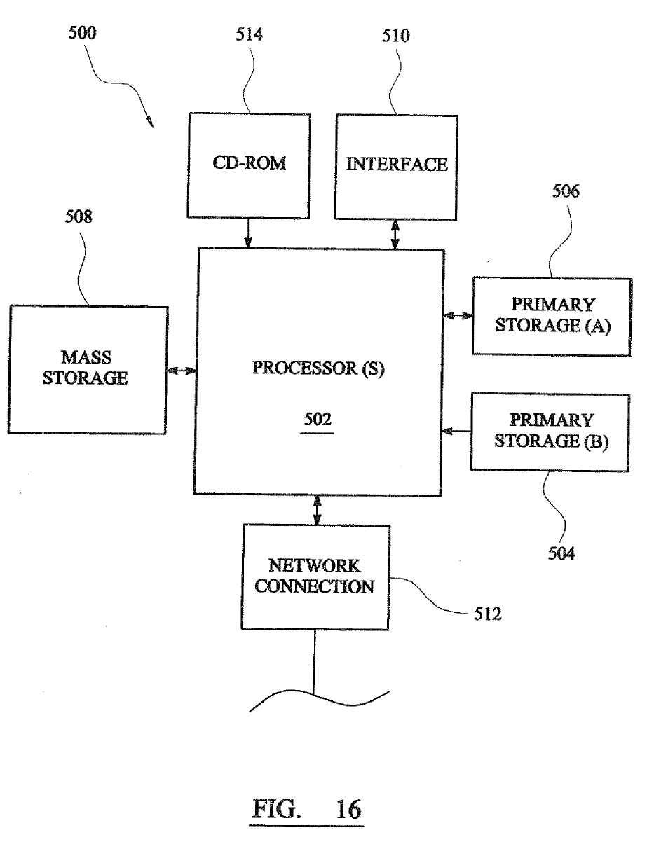

FIG. 15 shows an enlarged cross sectional view of a foot part of the guide shown in FIG. 14 in greater detail; and

FIG. 16 shows a schematic block diagram of a general purpose computer part of the computer aided surgical systems shown in FIGS. 6, 7 and 9.

Similar items in different Figures share common reference numerals unless indicated otherwise.

The present invention will be described with particular reference to an orthopaedic surgical procedure and in particular a femoral head resurfacing procedure. However, the present invention is not limited to such procedures and can be used in any procedure in which it is advantageous to be able to determine the position and/or orientation of a substantially linear axis or straight line, relative to a body part. For example, the jig can be used in orthopaedic, cranial, spinal, ENT (ear nose and throat), trauma, optical and other surgical and clinical procedures. Further, the invention is not limited to use with bones, but can be used in relation to any body parts, such as soft tissues, soft tissue structures, organs, the skin and any part of the animal or human body.

With reference to FIG. 1, there is shown a perspective view of a surgical jig 100, according to the present invention. The jig 100 includes a support 102 having a body section 104. The body 104 has a generally square cross-sectional shape and includes first 106, second 108, third 110 and fourth 112 generally rectangular shaped walls, First and second walls 106 and 110 provide a first proposed pair of walls. Second wall 108 and fourth wall 112 provide a second pair of proposed walls. Viewing apertures are provided in each wall so that the interior of the support is highly visualisable.

A foot member 114, 116, 118 and 120 are provided at each corner of the support although only three are visible in FIG. 1. An aperture is provided at the free end of each foot for receiving a pin in use to secure the jig to a body part. This provides a fastening mechanism for securing the jig to a body part.

Other fastening mechanisms can be provided. For example, the foot members can be made from a resiliently deformable material such that they are spring loaded and are shaped to clamp around a body part in a push fit or snap manner. Alternatively, the feet can be attached to the body part of the support by a sprung hinge so that the feet can be clamped around a body part to releasably secure the jig to the body part. In another fastening mechanism, a jubilee clip, or similar fastenable, band can be provided around the feet so to secure the jig to the body part, e.g. around the femoral head. The body part surface engaging parts of the feet can be formed so as to match to the anatomical shape of the body part to which they are to be secured, so as to provide a better fit and/or some degree of automatic positioning or alignment of the jig when it is mounted on the body part.

The jig 100 includes a first upper guide element 130 and a second lower guide element 180. The first and second guide elements and related drive mechanisms are similar in construction.

The first guide element 130 includes a guide channel 132 passing there through. As better illustrated in FIGS. 2A and 2B, the first guide channel 132 has a generally conical shape which flares along its longitudinal dimension. Guide channel 132 is a closed bore although in other embodiments, the guide channel can have an at least partially open configuration, such as a C or U shape in order to more easily accept an instrument or other auxiliary component. The second guide element includes a second guide channel 182 similar to guide channel 132 but inverted.

Guide element 130 has a first threaded bore passing through it which receives a first lead screw 134 and a second threaded bore, perpendicular to the first threaded bore, which receives a second lead screw 136 passing there through. Lead screws 136 and 134 act as carriages which bear the guide plate 130 and allow it to move by translation over a first plane, which is parallel to each of the lead screws and substantially perpendicular to the longitudinal axis of the jig 100. The second guide plate 180 is similarly borne by third and fourth mutually perpendicular lead screws 184 and 186 which are both parallel to a second plane which is also substantially perpendicular to the longitudinal axis of the jig and parallel to the first plane. Guide plate 180 similarly can be moved by the carriages provided by lead screws 184 and 186 to be translated over the second plane. An electric motor 138, 140, 188, 190 is operably connected to each lead screw as part of the drive mechanism. The drive mechanisms will be described in greater detail below with reference to FIG. 3. In alternate embodiments, motors are not used and instead a thumb wheel, or similar, is provided so that the lead screws can be manually rotated by a user to manually position the guide elements 130, 180 within the jig.

The general principle of operation of the jig will now be described with particular reference to FIGS. 2A and 2B. FIG. 2A schematically shows the first guide element 130 and drive mechanism parts and the lower guide element 180 and its drive mechanism. The first guide channel 132 and second guide channel 182 define between them a substantially straight line extending between them which defines the jig axis, illustrated by broken line 204. FIG. 2B illustrates a second configuration of he first and second guide elements in which motor 140 has been actuated to drive the guide element 130 to the right thereby changing the orientation of the jig axis 204 as defined by the first and second guide channels 132 and 182. Both the first guide plate 130 and the second guide plate 180 have two degrees of freedom in which they can move in parallel planes and so by operating the drive mechanisms, the position of the guide channels can be changed so as to define any jig axis orientation and position passing through the support 102.

The support 102 can be made of a plastics, stainless steel or any other suitable medically inert material.

The drive mechanism will now be described in greater detail with reference to FIGS. 3 and 4. FIG. 3 shows a schematic transverse cross-sectional view through either the upper or lower drive mechanism part of the jig. FIG. 4 shows a cross-sectional view along the longitudinal axis of the jig through the upper or lower drive mechanisms similarly. The lead screw 300 passes through a threaded bore in the guide element 302 and a free end of the threaded screw is pivotably received in a first bushing 304. An upper element 306 of the support frame has a rib 308 downwardly depending therefrom. A lower element 310 of the support frame has a further rib 312 upwardly depending therefrom. Ribs 308 and 312 are received within upper and lower grooves in the surface of bushing 304 which has a generally square or rectangular cross-section. This prevents the bushings from rotating in the slider channels. Ribs 308, 312 co-operate with the bushing such that the bushing can slide along the channel defined by the upper and lower members 306, 310 as further illustrated in FIG. 1.

A similar bushing 320 is provided on the opposite side of the support frame which similarly can slide along a ribbed channel in the opposite side of the support frame. The second bushing 320 has a bore passing there through which receives a drive shaft 322 of motor 138 which is mounted on bushing 320 by a support member 324. A similar drive mechanism 340 is provided perpendicular to the first drive mechanism 300 for both the upper and lower guide plates. Each bushing is made from a plastics material, such as polyethylene, acetyl and PEEL (polyether ether ketone). Also the inner surface of the slider channels can have a friction reducing surface, so as to provide a smooth bearing surface. Suitable materials include, e.g., polyethylene, acetyl and PEEK (polyether ether ketone).

Although the upper guide plate is illustrated in FIG. 4, the lower guide plate and associated drive mechanisms are configured similarly. When motor 138 is operated, lead screw 134 drives the guide plate 130 in a first direction, as illustrated by arrow 330 in the upper plane. The second drive mechanism 340 slides along the channel by virtue of the sliders provided on either side of the support frame. When motor 140 is operated, lead screw 136 causes the guide plate 130 to move in a second direction, perpendicular to the first direction, as illustrated by arrow 332. The first drive mechanism 300 slides and is translated with the guide plate. It will be appreciated that if both motors are driven at the same time, the guide plate will move diagonally.

By appropriate control of the motors, the guide plate 130 can be driven in translation to any position within the support frame. Hence the position of the guide channel 132 can also be moved over the upper plane. Similarly, the lower guide plate 180 can be moved over the lower plane and the lower guide channel 182 located at any position over the lower plane within the support frame. Hence by appropriately driving the upper and lower drive mechanisms, the upper and lower guide channels can be positioned to define any jig axis orientation and position lying within the support structure.

Although a particular lead screw drive mechanism has been described above, it will be appreciated that other drive mechanisms are possible. For example a pulley based mechanism could be used. Alternatively, a chain and cog based mechanism could be used. Also, although an electrical motor is described, other embodiments can use other motors, such as a pneumatic or hydraulic motor, and the drive can be provided by a manual source, such as a manually rotatable thumb wheel or lever. Therefore, other drive mechanisms are envisaged and any drive mechanism allowing two degrees of freedom so that the guide plate can be translated over a plane can be used. However, the described embodiment is a particular simple mechanism with a small number of parts and is relatively easy to miniaturise and to control to provide the accuracy required of the guide jig.

The use of the jig in a surgical procedure will now be described with particular reference to FIG. 5. FIG. 5 shows a flowchart 350 illustrating various steps carried out in a surgical procedure using the jig of the present invention. The flowchart is by way of illustration only and commonly known steps and procedures have been omitted. Further, other steps and procedures will precede and follow those particularly described as will be apparent to a person of ordinary skill in the art. However, these steps have not been described so as not to obscure the nature of the present invention. Further, some of the steps described can be optional and/or their order can be changed without affecting the efficacy of the invention, as will be apparent to a person of ordinary skill in the art. Flowchart 350 illustrates an orthopaedic surgical procedure and in particular a part of a hip replacement surgical procedure. The surgical procedure is begun at step 352 and at step 354 the hip is opened by the surgeon and at step 356 the hip is dislocated and rotated back out of position. The leg is rotated about the femoral axis to allow visualization of the femoral head by holding the ankle and knee and turning the leg in a medial direction.

As will be described in greater detail below, the jig of the present invention is particularly suited for use as a part of an image guided or computer aided surgery (CAS) system. Therefore when a computer aided surgical procedure is being carried out, at step 358 markers, trackable by a navigation system, are attached to landmark positions on the femur so that the navigation system can locate the position of the femur in space. Hence the location of the bone in free space is determined by the navigation system after step 358.

Image guided surgical (IGS) systems can display an image of captured body data, such as a CT scan, X-ray scan, ultrasound scan, to aid the surgeons in the carrying out of an operation. At step 360 it is determined whether a CT scan was previously carried out for the patient. If a CT scan was not carried out then at step 362, the surgeon uses a trackable probe to collect a series of points over the surface of the femur. The surgical system determines the position in space of the series of points which generates a net which defines the surface of the femur and so the surgical system now has access to data indicating both the location of the bone and the shape of the surface of the bone. If a CT scan was available, then the CT scan data can be used to generate the surface shape of the bone by identifying some key landmark points on the femur and then morphing the CT scan data in order to provide a three dimensional model of the bone from the CT scan data at step 364.

One hip replacement procedure includes the resurfacing of the femoral head. This procedure involves drilling a hole to receive a stem of a femoral head implant in which the hole preferably passes substantially along the axis of the femoral neck. Hence the surgeon wants to try and accurately determine the axis of the femoral neck. At step 366, the surgeon places the jig on the femoral head and generally aligns the jig with the femoral neck axis based on a visual inspection of the surgical site.

At step 368, the jig is controlled by a computer aided surgical system in order to automatically align the jig axis with the axis of the femoral neck. This process will be described in greater detail below. Once the jig has been aligned) the surgeon can carry out a manual check of the alignment of the axis defined by the jig with the axis of the femoral neck. For example, the surgeon can use a stylus or other piece of equipment in order to check whether mounting an implant using the determined axis would result in notching of the femoral neck which can lead to failure of the hip.

After the alignment has been checked at step 370, if necessary, at step 372, the surgeon can either fine tune the alignment by manually controlling the motors or can otherwise change the alignment axis, based on either experience or the circumstances of the particular surgical site. When the surgeon is satisfied with the axis defined by the jig, a drill bit is inserted through the guide channels and a pilot hole can be drilled in the head of the femur at step 374. In alternate embodiments, a cannulated drill can be used in which the actual drill bit passes through a tubular bushing which passes through the guide channels. When the pilot hole has been drilled, the jig is removed at step 376 and at step 378 the remaining surgical procedures are carried out to complete the operation.

With reference to FIG. 6, there is shown a computer aided surgical system 400 suitable for use in the surgical method illustrated in FIG. 5. The computer aided surgery (CAS) system 400 includes a tracking system 402, a jig 404 which has been adapted for use with a CAS system by trackable markers. A jig control and interface circuit 406 is also provided. The jig control circuit includes surgical connections for supplying control signals to the motor parts of the drive system of the jig 404. The jig control circuit 406 is also in communication with a computer 408 which is also in communication with the tracking system 402. Computer 408 stores or has access to various software modules for controlling the CAS system. For examples the computer 408 can include tracking software for determining the position of marked elements within the CAS system, surgical planning and navigation software and surgical procedure control software for controlling the operation of the jig during the surgical procedure.

In one embodiment, the computer sends data and control instructions to the jig interface circuit 406 which processes the data instructions to generate electrical control signals. In other embodiments, the computer can generate electrical control signals which are handled by the jig interface circuit 406 to generate the signals to control the operation of the electrical motors. The jig interface circuit 406 is shown as a separate functional component in FIGS. 6, 7 and 8 for the purposes of clarity of explanation only and can be provided as integral part of the computer system 408.

In the illustrated embodiments, an infrared wireless tracking system and infrared reflective markers are used. The tracking system 402 includes a source of infrared radiation. A first marker 410 is attached to the upper guide plate 130 and a second marker 412 is mounted on the second guide plate 180. Each of the markers comprises three spheres with a highly infrared reflective surface mounted in a triangular configuration to provide a marker detectable by the tracking system, also commonly referred to as a “star”. The tracking system 402 includes first and second infrared detectors 414, 416 which are offset from one another and survey the surgical site to detect the infrared radiation reflected from the markers.

In other embodiments, other tracking systems can be used. For example the markers can be wireless or wired. Various types of energy can be used, such as acoustic or electromagnetic radiation. For example and ultra sound based system can be used, and electromagnetic radiation in parts of the spectrum other than infra red can be used, e.g. radio frequency and microwave. Also, the markers can be passive ones that reflect radiation, or active ones that generate radiation.

Tracking software processes the infrared images collected by the tracking system to determine the position in space of the markers. Each marker is independently recognisable by the tracking system and once identified, the position of the guide channel can be determined from the position of the marker using information previously registered with the tracking software. Hence, the tracking system and computer can determine the locations in space of the guide channels and therefore determine the position and orientation of the axis defined by the current position of the guide channels as the tracking system monitors the movement of the guide plates.

As illustrated in FIG. 6, the jig 404 is mounted on a femoral head 420 having a femoral neck 422 attached thereto. The femoral neck has a neck axis illustrated by dashed line 424. The jig has a jig axis indicated by dashed line 426 defined by the current position of the guide channels.

During a planning stage of the operation, which may be pre-operative or intra-operative, the surgeon can define the axis of the body part with which he wishes to align the jig axis. The pre-determined axis can be any axis relative to a body part and does not necessarily need to coincide with an axis of a physical body part. However, in the presently described example, the predetermined axis corresponds substantially to the axis of the femoral neck 424.

In a pre-operative planning approach, the surgeon can review scans of the femoral head and neck, e.g. CT, X-ray or ultrasound scans, using the planning software of the CAS system and can identify from the scans the axis of the femoral neck. Hence during the planning stage the surgeon defines the predetermined body part axis in the image data. At step 364 in FIG. 5, the body scan data is morphed to the collected body part data. The position of the body part is registered with the CAS system at step 358 and so the position of the predetermined axis is mapped on to the body part position during the morphing step 364.

In an intra-operative approach, the surgeon can identify the direction of the axis to the CAS system, e.g. using a pointer during step 362, in a sub-procedure in which the CAS system detects the position of at least two points to define the predetermined body part axis. As the position of the body part has been determined by the CAS system at 358, the position of the predetermined axis is also available from the CAS system.

Operations to be carried out by the CAS system during the auto-alignment step 368 will now be described in greater detail with reference to FIG. 8. FIG. 8 shows a flowchart 430 illustrating processing steps carried out by the computer system 408 which are implemented by appropriate software. The auto-alignment procedure is called and initiates at step 432. At step 434, data representing the current position of the top and bottom guide channels is obtained from the tracking system. From this data the current position of the jig axis can be calculated. At step 436, the program obtains data representing the position of the predetermined body axis from the surgical planning software and also data indicating the position of the predetermined body axis in the reference system of the tracking system as obtained from the planning software. The program then determines at step 436 the positional difference in the reference frame of the tracking system between the current jig axis and the predetermined body axis.

If it is determined at step 438 that the jig axis and body axis are not aligned, as will likely be the case initially, then process flow proceeds to step 440. At step 440, the program operates on the jig axis positional data and body axis positional data to determine the appropriate control signals to send to the jig so as to reduce the positional separation between the body axis and jig axis. Electrical control signals are output from the jig interface circuit 406 to the motors which move the guide plates appropriately. As the guide plates move, the tracking system 402 detects the current position of the guide plates and process flow loops 444 and the program, in a first situation, determines at step 434 the current position of the jig axis. Again at step 436 the difference between the jig axis and body axis is determined and again at step 438 it is determined whether the jig axis and body axis are currently aligned. If not, then process flow continues to loop until at step 438 it is determined that the jig and body axis are aligned. Once the jig and body axis are determined to be aligned, then the auto-alignment procedure terminates at step 446. Hence, the tracking system provides a feedback loop in the CAS system to provide automatic alignment of the jig axis and body axis.

FIG. 7 shows a CAS system 440 similar to that shown in FIG. 6, but in which the jig is marked differently. In this embodiment, a marked instrument 442 is located in the guide channels and so follows the orientation of the jig axis as the guide plates are moved. The instrument 442 includes a wirelessly trackable marker 444 the instrument and marker are registered with the tracking system and the marker provides a signature for the instrument which is recognised by the tracking system. There is a known relationship between the position of the marker and the shape and form of the instrument. Hence by tracking the position of the marker, the CAS system can determine the orientation of the jig axis which is co-linear with the instrument 442.

The method of operation of CAS 440 is similar to that described previously with reference to FIG. 8.

FIG. 9 shows a further embodiment of the CAS system in which the jig is configured differently for automatic alignment. In this embodiment, the jig 454 includes stepper motors. The jig 454 bears a wirelessly trackable marker 452 which is recognisable by the tracking system in order to determine the position in the reference system of the tracking system of the jig. However, in this embodiment, rather than tracking the movement of the plates, signals from the stepper motors are used to determine the positions of the plates relative to the jig. As the position of the jig itself is known, the position of the plates within the reference system of the tracking system can be determined.

The jig interface circuit 460 is different in this embodiment and includes circuitry to receive signals from the stepper motors which indicate their position and to provide signals to the computer system 408 indicating the position of the stepper motors relative to the jig. Alternatively, the interface circuitry 460 can include a processing device which transmits positional data to the computer 408. Depending on the embodiment of the jig interfacing circuit 460, computer 408 either sends electrical signals to control the operation of the stepper motors or data or instructions interpretable by the interface circuit to generate the necessary control signals to operate the stepper motors.

FIG. 10 shows a flowchart 470 illustrating the operations carried out by an auto-alignment program routine during the auto-alignment step 368 of the method as illustrated in FIG. 5. The program initiates at step 472 when the auto-alignment procedure is called. The program obtains data from the tracking system representing the position of the jig in the reference system of the tracking system at step 474. Then at step 476, the program obtains data indicating the initial positions of the upper and lower guide plates from the stepper motors via interface circuit 460. At step 478, the program determines the initial position of the jig axis using the data representing the initial position of the guide plates, as there is a fixed relationship between the position of the guide plates and the guide channels which define the jig axis. At step 480, the program determines the difference between the initial jig axis position and the body axis position in a manner similar to that described above the reference to FIG. 8. Then at step 482, the program determines the adjustments required to the initial guide plate positions in order to align the jig axis with the body axis and generate appropriate stepper motor control or data signals which are output to the interface circuit 460. The interface circuit 460 applies control signals to the stepper motors to drive the guide plates so that the jig axis is aligned with the predetermined body part axis.

In one embodiment, no feedback is used to determine the eventual jig axis position and the process can terminate at this time. In an alternative embodiment, the interface circuit 460 receives signals from the stepper motors which are processed to determine the position of the stepper motors and the positional data is returned to the computer 408 and at step 484, the program determines whether the jig axis and body axis are aligned. If they are determined to be aligned, then the process can terminate at step 486. if not, then process flow returns 488 and the preceding operations are repeated until it is determined that the jig axis and body axis have been aligned.

With reference to FIG. 11 there is shown a further embodiment of a surgical jig 600 according to the invention. The jig includes a support 602 comprising a shaft 604 with a circular cross-section and having a longitudinal axis. The shaft has a first, upper free end and a lower end which is attached to a lower portion 606. The lower portion has a generally inverted U-shape. A circular cross-section pin or peg extends between the free ends of the lower part 606. A base member 608 is ?? around the peg so as to be able to pivot about a longitudinal axis of the peg, as illustrated by line 610. Base member 608 has an aperture 612 there through for receiving a fastening in use for securing the base 608 to a bone. Base member 608 extends around the peg and an upper and lower free part of member 608 can be drawn together by a fastener to clamp the base member 608 around the peg so as to prevent pivoting of the lower portion 606 of the support relative to the base member 608.

FIG. 15 shows a schematic cross-section through the lower part of the support and also illustrates protrusions 614, 616 on a lower bone engaging face of base member 608. These protrusions are in the forms of spikes which provide bone engaging members which in use can penetrate the bone so as to securely locate the base and prevent the base from rotating relative to the bone surface. As also illustrated in FIG. 15, a fastener, such as a bone screw 618 can be introduced into aperture 612 and screwed into the bone thereby imparting a clamping action, as illustrated by arrow 620 about the peg part of the lower part of the support to prevent pivoting of the support relative to the base 608. Hence the base provides a foot to the support by which it can be connected to the bone in use.

Returning to FIG. 11, the support includes a first upper arm 622 and a second lower arm 624. The upper arm and lower arm are each pivotally connected to the shaft 604 so as to be rotatable about the longitudinal axis of shaft 604. The upper arm and lower arm are longitudinally spaced along the longitudinal axis of shaft 604. The first arm 622 bears a first guide element 626 at a distal free end thereof. The second arm bears a second guide element 628 at a distal free end thereof Each guide element has a guide channel there through for receiving a guide or instrument 630 there through and between them define a substantially linear jig axis. The first and second arms are each extendable along a longitudinal axis of the arms so that the length of each arm can be varied independently.

FIG. 13 shows an enlarged view of the detail of the construction of each of the first and second arms 640. As illustrated, each arm includes a housing 642 rotatably attached to shaft 604 and the arm includes two separable parts 644 and 646. A threaded bore extends through arm part 644 and receives a threaded shaft 648 therein having one end rotatably engaged with the second arm part 646. A knurled handle 650 is attached to the shaft and can be rotated, either manually or by being driven by a motor in order to change the length of the arm by driving second arm part 646 away or toward first arm part 644.

Shaft 604 includes a gear 652 mounted thereon. Housing 642 includes a further bore within which a shaft of a second drive element 654 is rotationally received. Drive element 654 has a worm gear 656 engaged with gear 652 and a handle 658 by which the worm gear can be rotated either manually or by being driven by an attached motor. By rotating drive element 654, the arm is caused to pivot about the longitudinal axis of shaft 604. A similar drive arrangement 640 is provided for both the upper and lower arm.

As illustrated in FIG. 12, the upper and lower guide elements can move throughout a sector 660 of an annular space in two parallel planes by moving along radial directions and being pivoted through angular directions. Hence the upper and lower guide elements can between them define a substantially linear guide axis.

FIG. 14 shows the surgical jig 600 fastened in use to the head of a femur 662. In use, the practitioner can initially position the guide jig close to where the axis of the femur is believed to be. Then a fastener, such as bone screw 618 is introduced into aperture 612 in the base member 608 and the jig is initially secured to the femur 662. The support of the jig can be pivoted about the base to more closely align the jig with the intended axis. Once the jig position has been approximately set, the screw 618 can be tightened further thereby clamping the shaft 604 relative to the base to prevent further motion. Also, the action of the screw 618 in the bone causes spikes 614, 616 to be driven into the surface of the bone to prevent the base rotating relative to bone screw 618.

Then, using methods similar to those described above, the drive elements 658 and 650 can be operated to adjust the position of the upper and lower guide members 626, 628 until the guide axis 664 defined by the upper and lower guide elements has been aligned with an intended axis of the body part. It may be that the intended axis defined by the guide axis 664 is different to an axis of the body part, as illustrated by line 666 which would have been arrived at from inspection of the body part only without the use of a guide jig.

The further embodiment of the jig can be adapted for use with a computer aided surgical system and in computer aided surgical procedures as described above in relation to the first embodiment of the jig.

Generally, aspects of the present invention employ various processes involving data stored in or transferred through or processed by one or more computer systems. Embodiments of the present invention also relate to an apparatus and computer program code for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

FIG. 11 illustrates a typical computer system that, when appropriately configured or designed, can provide the computer part 408 of the computer aided surgical system aspect of the invention. The computer system 500 includes any number of processors 502 (also referred to as central processing units, or CPUs) that are coupled to storage devices including primary storage 506 (typically a random access memory or RAM), primary storage 504 (typically a read only memory, or ROM). CPU 502 may be of various types including microcontrollers and microprocessors such as programmable devices (e.g., CPLDs and FPGAs) and unprogrammable devices such as gate array ASICs or general purpose microprocessors. As is well known in the art, primary storage 504 acts to transfer data and instructions uni-directionally to the CPU and primary storage 506 is used typically to transfer data and instructions in a bidirectional manner. Both of these primary storage devices may include any suitable computer-readable media such as those described above. A mass storage device 508 is also coupled bi-directionally to CPU 502 and provides additional data storage capacity and may include any of the computer-readable media described above. Mass storage device 508 may be used to store programs, data and the like and is typically a secondary storage medium such as a hard disk. For example different applications used in the CAS system may be stored on the mass storage device, such as navigation and planning applications. It will be appreciated that the information retained within the mass storage device 508, may, in appropriate cases, be incorporated in standard fashion as part of primary storage 506 as virtual memory. A specific mass storage device such as a CD-ROM 514 may also pass data uni-directionally to the CPU.

CPU 502 is also coupled to an interface 510 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 502 optionally may be coupled to an external device such as a database or a computer or telecommunications network using an external connection as shown generally at 512. With such a connection, it is contemplated that the CPU might receive information from the network, or might output information to the network in the course of performing the method steps described herein. For example image files for scans carried out on the patient may be distributed over a network ad the CAS system may retrieve the relevant image files for use by the local navigation and/or planning applications.

Although the above has generally described the present invention according to specific processes and apparatus, the present invention has a much broader range of applicability. In particular, aspects of the present invention is not limited to any particular kind of surgical procedure or tracking or location mechanism and can be applied to virtually any surgical procedure where accurate determination, manual or automated, of the position and/or orientation of an axis or part of an axis relative to a body part would be beneficial. One of ordinary skill in the art would recognize other variants, modifications and alternatives in light of the foregoing discussion.

1. A surgical jig for defining an axis relative to a body part, the jig comprising:
a support;
a first guide element having a first guide channel, the first guide element being mounted on the support and being translatable over a first plane; and
a second guide element having a second guide channel, the second guide element being mounted on the support and being translatable over a second plane, the second plane being parallel to the first plane, and wherein the first guide channel and second guide channel between them define a substantially linear jig axis.

2. The jig of claim 1, further comprising a drive mechanism operable to move one or both of the first guide element and the second guide element.

3. The jig of claim 1, wherein the support is a frame.

4. The jig of claim 2, wherein the drive mechanism includes first and second carriers bearing the first guide element, the first and second carriers being disposed parallel to the first plane and being perpendicular to each other.

5. The jig of claim 4, wherein the drive mechanism includes a motor actuable to drive the carrier to control the position of the first guide element over the plane.

6. The jig of claim 4, wherein the support includes a first pair of opposed sides, each side including a slider and a second pair of opposed sides, perpendicular to the first pair of opposed sides, wherein each side includes a slider, and wherein the first carriage extends between the sliders of the first pair of sides and the second carriage extends between the sliders of the second pair of sides.

7. The jig of claims 4, wherein the drive mechanism includes third and fourth carriers bearing the second guide element, the third and fourth carriers being disposed parallel to the second plane and being perpendicular to each other.

8. The jig of claim 7, wherein the drive mechanism includes a motor actuable to drive the carriers to control the position of the second guide element over the plane.

9. The jig of claim 7, wherein the first pair of opposed sides, each include a further slider and the second pair of opposed sides each include a further slider, and wherein the third carriage extends between the further sliders of the first pair of sides and the fourth carriage extends between the sliders of the second pair of sides.

10. The jig of claim 6, wherein each slider includes a guide track having a bushing slidably mounted therein and wherein the ends of the carriers are each received in a respective bushing.

11. The jig of claim 4, wherein each carrier is a lead screw.

12. The jig of claim 4, wherein each carrier is independently drivable.

13. The jig of claim 12, further comprising a separate motor for driving each carrier.

14. The jig of claim 13, wherein each motor is an electric motor.

15. The jig of claim 13, wherein each motor is a stepper motor.

16. The jig of claim 1, further comprising a first marker detectable by a tracking system.

17. The jig of claim 16, further comprising a second marker detectable by a tracking system, the second marker being attached to the second guide element and wherein the first marker is attached to the first guide element.

18. The jig of claim 16, further comprising an instrument passing through the first guide channel and second guide channel and wherein the first marker is attached to the instrument.

19. The jig of claim 1, wherein the support includes a plurality of feet engagable with a surface of the body part.

20. The jig of claim 19, wherein the plurality of feet can be clamped about the body part to secure the jig to the body part.

21. The jig of claim 1, further comprising a first arm by which the first guide element is connected to the support and a second arm by which the second guide element is connected to the support.

22. The jig of claim 21, wherein the first and second guide arms are spaced along a longitudinal axis of the support and are each pivotally connected to the support and can pivot about the longitudinal axis of the support.

23. The jig of claim 21, wherein the first and second arms are each extendable along a longitudinal axis of the arm.

24. The jig of claims 21, further comprising a base member pivotally attached to the support, and wherein the base member includes a formation for receiving a fastener to secure the guide to a bone.

25. The jig of claim 24, wherein a part of the support is journalled within the base member and wherein the base member can clamp around the part of the support to prevent relative movement between the support and base member when secured to the bone by the fastener.

26-40. (canceled)
